(19) United States
(12) Patent Application Publication
(10) Pub. No.: US 2013/0217066 A1
(43) Pub. Date: Aug. 22, 2013

(54) PIGMENT PRODUCTION BY LATEX-EMBEDDED MICROORGANISMS

(71) Applicants: Chris M. Yeager, Los Alamos, NM (US); Garriet W. Smith, Aiken, SC (US); Magdalena Piskorska, North Augusta, SC (US)

(72) Inventors: Chris M. Yeager, Los Alamos, NM (US); Garriet W. Smith, Aiken, SC (US); Magdalena Piskorska, North Augusta, SC (US)

(73) Assignees: University of South Carolina, Columbia, SC (US); Savannah River Nuclear Solutions, LLC, Aiken, SC (US)

(21) Appl. No.: 13/398,082

(22) Filed: Feb. 16, 2012

(51) Int. Cl. C12P 1/00

(52) U.S. Cl. 435/41

(57) ABSTRACT

A process is provided for the extraction of pigments from immobilized microorganisms. Pigment producing cells are immobilized and dried in a binder matrix, and pigments are recovered from the cells by liquid rinsing. Following release of the pigments, cellular metabolism of the microorganisms is revived in culture medium and pigments are regenerated. Pigments are then recovered from the immobilized cells by successive rounds of dehydration/rinsing/cell recovery.

### STATEMENT AS TO RIGHTS TO INVENTIONS MADE UNDER FEDERALLY SPONSORED RESEARCH AND DEVELOPMENT

This invention was made with Government support under Contract No. DE-AC09-08SR22470 awarded by the United States Department of Energy. The Government has certain rights in the invention.

### FIELD OF THE INVENTION

This invention is directed towards a method of recovering pigments from microorganisms such as cyanobacteria.

Many of these cyanobacteria are characterized by the presence of pigments in additional to chlorophylls, which are responsible for the organisms color. Some of these pigments are known as phycobiliproteins and they are useful as food dyes. The phycobiliproteins are characterized by an intense color, high solubility in water, and stability over a broad range of pH. Accordingly, applications for these natural pigments in food, beverages, and cosmetics, have proven useful.

### BACKGROUND OF THE INVENTION

This invention relates to extraction of pigments from microorganisms. Pigment extraction typically involves harvesting a biomass from a bioreactor or open impoundment. Pigment is typically extracted by disruption of the cells, where upon the pigment is recovered. However, recovery of the pigment results in the death of the harvested cells. The remaining biomass must be discarded as a waste product or utilized as some type of secondary product. In a conventional pigment recovery process, there must be multiple bioreactors for a continuous supply of microorganisms in order to produce pigment on a regular and repetitive basis.

Further, traditional extracting techniques often require large volumes of organic solvents. The handling of organic solvents can be logistically difficult and potentially hazardous depending upon the nature of the solvents utilized. Accordingly, there remains additional room for variations and improvements with respect with recovery of pigments from microorganisms.

### SUMMARY OF THE INVENTION

It is one aspect of one of the present embodiments to provide for a process of extracting a pigment from a microorganism comprising the steps of:

suspending a sample of pigment containing cells in a latex, thereby, providing a mixture of a latex and pigment containing cells;

apply the mixture to a substrate;

allowing mixture to dry on the substrate, and thereby providing an immobilized population of pigment producing cells;

placing said immobilized population of cells in a liquid cell culture medium;

incubating said immobilized population of cells for a time sufficient to produce a desired quantity of pigments;

drying the immobilized population of cells; rinsing the immobilized population of cells, thereby releasing a pigment from the cells into a rinse;

recovering pigments from the rinse;

incubating further the immobilized cells in an incubation media for a time sufficient to produce additional pigments; and, repeating the drying, rinsing and recovery step set forth above.

It is a further aspect of at least one embodiment of the present invention to provide for a process of extracting a pigment from a microorganism wherein the process of drying immobilized population of cells further includes maintaining the immobilized population of cells at a relative humidity of about 50% for an interval of at least about 24 hours.

It is a further aspect of at least one embodiment of the present invention to provide for a process of extracting a pigment from a microorganism comprising the steps of:

suspending a sample of pigment containing cells in a binder, thereby, providing a mixture of a binder and pigment containing cells;

applying the mixture to a substrate;

allowing mixture to dry on the substrate, and thereby providing an immobilized population of pigment producing cells;

placing said immobilized population of cells in a liquid cell culture medium;

incubating said immobilized population of cells for a time sufficient to produce a desired quantity of pigments within the cells;

drying the immobilized population of cells;

rinsing the immobilized population of cells, thereby releasing a pigment from the cells into rinse; and

recovering pigments from the rinse.

It is a further aspect of at least one embodiment of the present invention to provide for the process of extracting a pigment from a microorganism wherein pigments are released from a dried, immobilized population of cells by liquid rinsing.

It is a further aspect of at least one embodiment of the present invention to provide for a process of extracting a pigment from an immobilized population of microorganisms wherein the microorganisms are Cyanobacteria.

These and other features, aspects, and advantages of the present invention will become better understood with reference to the following description and appended claims.

### BRIEF DESCRIPTION OF THE DRAWINGS

A fully enabling disclosure of the present invention, including the best mode thereof to one of ordinary skill in the art, is set forth more particularly in the remainder of the specification, including reference to the accompanying drawings.

FIG. 1 is a diagram of a process of extracting pigments from an immobilized population of cells in accordance with one embodiment of the present invention.

### DESCRIPTION OF THE PREFERRED EMBODIMENT

Reference will now be made in detail to the embodiments of the invention, one or more examples of which are set forth below. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used on another embodiment to yield a still further embodiment. Thus, it is intended that the present invention cover such modifications and variations as come within the scope of the appended claims and their equivalents. Other objects, features, and aspects of the present invention are disclosed in the following detailed description. It is to be understood by one of ordinary skill in the art that the present discussion is a description of exemplary embodiments only and is not intended as limiting the broader aspects of the present invention, which broader aspects are embodied in the exemplary constructions.

In describing the various figures herein, the same reference numbers are used throughout to describe the same material, apparatus, or process pathway. To avoid redundancy, detailed descriptions of much of the apparatus once described in relation to a figure is not repeated in the descriptions of subsequent figures, although such apparatus or process is labeled with the same reference numbers.

According to the present invention, it has been found that sustained pigment production and extraction from the immobilized microorganisms can be obtained in the manner that does not involve loss or destruction of the microorganism. The process involves the immobilization of pigment producing organisms in a latex coating followed by subsequent pigment extraction through hydration of the organisms bound in the dried latex coating. Following extraction, new pigments can be generated in rewetted latex immobilized cells, which allows for multiple cycles of pigment extraction using the same cells.

Pigments associated with microorganisms include carotenoids, flavonoids and tetrapyrroles, which include chlorophylls and phycobiliproteins. It has been found that use of biocompatible latex coatings provide a stable matrix useful for the immobilization of microbes. The resulting solid support matrix is ideal for photosynthetic microorganisms because the support matrix provides for an increase of surface area for incident light and photosynthesis; facilitates high rates of gas diffusion compared to a liquid-based incubation systems; and, maintains cell longevity by protecting the microorganisms from mechanical degradation, release from the substrate, and contamination. Additionally, the use of an immobilizing matrix for the cells substantially prevents cell growth and division. As a result, CO₂ fixation by photosynthesis is not diverted to cell division or growth so that a higher percentage of the fixed CO₂ can be converted to the production of pigments.

The phycobiliproteins associated with certain microbial organisms are released into a surrounding medium upon hydration of the matrix/coating. While the pigments are released, the cells remain entrapped within the coatings and can be induced to produce additional photosynthetic pigments when incubated in culture.

The mixture of pigment producing cells and latex can be applied to polyester sheets. Prior to latex addition, bacterial cells are harvested by centrifugation from growth medium and mixed with the indicated amount of glycerol and sucrose. The latex emulsions for coatings are prepared based on the formulation ratio of 1.2 g wet cell weight, 350 μl of 1.7 M sucrose, 150 μl of 100% glycerol, and 1 ml of latex. Coatings are prepared as strips using a template design consisting of a glass support, a 125-μm thick polyester sheet (DuPont Melinex 454, Tekra Corp, N.J.), and an adhesive vinyl mask (84 μm thick, Con-Tact, Stamford, Conn.). The polyester sheet was pre-cut with parallel lines separated by 1 cm to define the width of each strip, and attached to a glass support covered with double-sided Scotch tape (strips perpendicular to the tape). An adhesive vinyl mask with a pre-cut rectangle (5 cm long to define the length of each strip; modified to 3.5 cm where indicated) may be placed on top of the polyester so that its parallel pre-cut lines are in the center of the mask opening. The width of the mask opening may be determined by the number of polyester strips plus an additional 0.5 cm on each end for template design details.

The latex/cell formulation is transferred onto an assembled polyester template where it is spread across surface. A 26-wire wound Mayer rod may be drawn by hand down the template mask in order to spread the formulation. The coatings are allowed to dry in a glove box. Each individual polyester strip (1×5 cm, unless otherwise noted) is “painted” with embedded cells and is then hydrated with culture medium. While the protocol described above has been found suitable for the studies and results presented therein, it is recognized that variations to the process and materials can be utilized. For instance, variations in relative amounts of cells to the latex matrix are possible as well as the use of other binder materials such as polyacrylamide instead of latex or latex in combination with other suitable binder materials which may include polyacrylamide. Similarly, the lab techniques using small polyester strips can be expanded to larger sheets that can be used with various vats and immersion chambers for the appropriate incubation and pigment recovery steps.

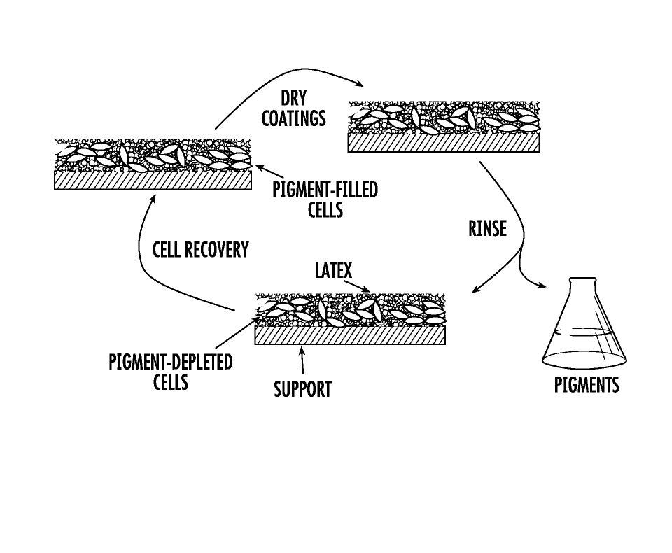

As best seen in reference to FIG. 1, the mixture of pigment containing cells and latex can be applied to a substrate such as a polyester sheet as described above. Depending upon the pigment content of the initial cell culture used to perform the mixture, the pigment extraction through rehydration can be done once the latex mixture has sufficiently cured to the substrate. Alternatively, it may be preferred to incubate the cells by infusion with media for a sufficient time interval until pigment production is observed. Following incubation with media, the substrate containing the immobilized cells may be removed from the incubation media and stored in a 50% relatively humidity environment for a time interval of about 24 hours. Following the dry incubation step, the substrate and cells are placed in a rinse media, which may be a liquid growth media. Depending upon the pigment, species, and the length of drying, the pigments are released into the culture media by diffusion process over the course of several hours. The pigments may thereafter be separated from the culture media using any number of conventional purification techniques. Following removal of the pigments, the immobilized population of cells may be further incubated with medium for a time interval of a duration to allow the immobilized population of cells to synthesize a sufficient amount of pigment, at which point the drying and pigment removal steps may be repeated.

It has been found that multiple cycles of pigment extraction and cell recovery are possible. For instance, 5 successive rounds of pigment production and extraction were achieved using the above technology with the cyanobacterium Anabaena PCC 7120. No appreciable loss of cells or decrease in pigment production was noted. The resulting phycocoyanin released by the immobilized organisms was food grade quality pigment without additional purification.

The pigment extraction processes and protocol can be varied. For instance, while a 10-day interval cell recovery in between pigment extractions has been found useful for the Nostocales organisms tested, it is believed that the incubation period can vary significantly while still providing useful yields of quality pigments.

In addition, the amount of time the immobilized organisms spend under drying conditions may also affect the amount of pigment recovery and/or the time interval needed for recovery. It is believed that one having ordinary skill in the art can evaluate various storage conditions to further optimize pigment production keeping in mind that different species may respond differently to varying storage conditions or to length of time in which cell recovery in the presence of incubation medium is provided. For example, differences in pigment release and cell recovery have been noted when cells are dried at relative humidity levels ranging from 0-60%.

The above described process and materials provides for repeated use of cultured microorganisms to recover pigments there from. The process results in improved economy in that the cell cultures are not destroyed during the extraction process and that the same cells can be used for multiple cycles of pigment recovery.

Although preferred embodiments of the invention have been described using specific terms, devices, and methods, such description is for illustrative purposes only. The words used are words of description rather than of limitation. It is to be understood that changes and variations may be made by those of ordinary skill in the art without departing from the spirit or the scope of the claims. In addition, it should be understood that aspects of the various embodiments may be interchanged, both in whole, or in part. Therefore, the spirit and scope of the invention should not be limited to the description of the preferred versions contained therein.

1. A process of extracting a pigment from a microorganism comprising the steps of:
suspending a sample of pigment producing cells in a latex, thereby, providing a mixture of a latex and pigment containing cells;
apply the mixture to a substrate;
allowing mixture to dry on the substrate, and thereby providing an immobilized population of pigment producing cells;
placing said immobilized population of cells in a liquid cell culture medium;
incubating said immobilized population of cells for a time sufficient to produce a desired quantity of pigments within the cells;
drying the immobilized population of cells;
rinsing the immobilized population of cells, thereby releasing a pigment from the cells into rinse; and
recovering pigments from the rinse.

2. The process according to claim 1 comprising the additional steps of incubating further the immobilized cells in an incubation media for a time sufficient to produce additional pigments; and, repeating the drying, rinsing and recovery step set forth above.

3. A process of extracting a pigment from a microorganism comprising the steps of:
suspending a sample of pigment producing cells in a binder, thereby, providing a mixture of a binder and pigment containing cells;
applying the mixture to a substrate;
allowing mixture to dry on the substrate, and thereby providing an immobilized population of pigment producing cells;
placing said immobilized population of cells in a liquid cell culture medium;
incubating said immobilized population of cells for a time sufficient to produce a desired quantity of pigments within the cells;
drying the immobilized population of cells;
rinsing the immobilized population of cells, thereby releasing a pigment from the cells into rinse; and
recovering pigments from the rinse.

4. The process according to claim 3 wherein said binder is a latex.

5. The process according to claim 3 wherein said binder further comprises a mixture of a latex and a polyacrylamide.
